(19) United States
(12) Patent Application Publication
(10) Pub. No.: US 2022/0409097 A1
(43) Pub. Date: Dec. 29, 2022

(54) Joint Axis Direction Estimation

(71) Applicant: McLaren Applied Technologies Limited, Woking (GB)

(72) Inventors: Matthew A. Gaskell, London (GB); Nicholas H. Reddall, London (GB)

(21) Appl. No.: 17/754,964

(22) PCT Filed: Oct. 15, 2020
(86) PCT No.: PCT/IB2020/059718

§ 371 Date: Apr. 18, 2022

(30) Foreign Application Priority Data
Oct. 18, 2019 (GB) 1915138.0

(51) Int. Cl. A61B 5/11; A61B 5/00

(52) U.S. Cl. CPC A61B 5/1114; A61B 5/4528; A61B 5/1121; A61B 5/6801; A61B 2560/0223

(57) ABSTRACT

A method for calibrating respective estimated joint axis directions for each of a pair of body mounted sensors, one of the pair of sensors being located to each side of the joint comprising a joint axis, the sensors each calculating a pitch angle about respective first sensor axes and a roll angle about respective second sensor axes, the first and second sensor axes together with a third sensor axis orthogonal to the first and second sensor axes forming a sensor frame, the method comprising: receiving orientation data for each of the two sensors, the orientation data being associated with at least two different poses of the joint for each of the two sensors and the orientation data comprising the pitch angle and the roll angle of the sensor for each pose; calculating a sensor frame estimated gravity vector for each pose associated with each sensor based on the pitch and roll angles for each pose associated with each sensor and a gravity vector running along a vertical direction; and determining the estimated joint axis directions for the joint axis, relative to the first and second sensor axes, for each sensor that minimise a loss function concerning projections of each sensor frame estimated gravity vector for each pose associated with each sensor on to the estimated joint axis direction for the respective sensor.

### CROSS-REFERENCE TO RELATED APPLICATIONS

This application claims the benefit of priority from United Kingdom patent application no. 1915138.0, filed Oct. 18, 2019, which is incorporated by reference herein in its entirety.

### DETAILED DESCRIPTION

This invention relates to a method for calibrating respective estimated joint axis directions for each of a pair of body mounted sensors and a device configured to implement the method.

There is a growing popularity for devices that measure movement. These sensing devices could be in the form of wearable devices that measure movement of a user, a smartphone that is carried by the user to measure movement of the user or moveable devices that can generally sense movement, for instance video game controllers or sensors attached to industrial equipment. In particular, wearable devices can be utilised to track motion of a human or other animal and, in particular can be used to monitor the motion of a specific joint.

These sensing devices may include a satellite positioning sensor which can sense the location of the device, and one or more motion sensors which sense motion and/or orientation of the device. These motion sensors may include one or more of an accelerometer, a gyroscope, a magnetometer, a compass and a barometer. Measurements taken by the sensors can be used to provide information about the joint.

The sensing devices can be attached to the body about a joint to provide data on the movement of that joint. The joint usually moves about a joint axis at the centre of the joint. It is difficult to attach the sensor devices to the body in a way that means the sensed rotation axes of the sensor device align with the movement axis or axes of the joint. In some cases, due to the shape of the body, it is impossible for the sensed rotation axes to be made to align with the movement axis or axes of the joint even with very careful positioning. This difference between how the sensor senses movement along certain axes and how the joint actually moves can lead to inaccuracies in the measurement of the movement of the joint.

It would therefore be desirable for there to be a method of correcting for the differences between the sensed rotations of the sensing devices and the actual movement of the joint.

According to a first aspect of the present invention there is provided a method for calibrating respective estimated joint axis directions for each of a pair of body mounted sensors, one of the pair of sensors being located to each side of the joint comprising a joint axis, the sensors each calculating a pitch angle about respective first sensor axes and a roll angle about respective second sensor axes, the first and second sensor axes together with a third sensor axis orthogonal to the first and second sensor axes forming a sensor frame, the method comprising: receiving orientation data for each of the two sensors, the orientation data being associated with at least two different poses of the joint for each of the two sensors and the orientation data comprising the pitch angle and the roll angle of the sensor for each pose; calculating a sensor frame estimated gravity vector for each pose associated with each sensor based on the pitch and roll angles for each pose associated with each sensor and a gravity vector running along a vertical direction; and determining the estimated joint axis directions for the joint axis, relative to the first and second sensor axes, for each sensor that minimise a loss function concerning projections of each sensor frame estimated gravity vector for each pose associated with each sensor on to the estimated joint axis direction for the respective sensor.

The gravity vector may run along the vertical direction in a negative direction. The gravity vector may run along the vertical direction in an upward direction.

Calculating the sensor frame estimated gravity vector for each pose associated with each sensor may comprise forming a rotation matrix for each pose associated with sensor using the pitch and roll angles for each pose associated with each sensor. Forming a rotation matrix for each pose associated with each sensor using the pitch and roll angles for each pose associated with each sensor may comprise assuming the rotation about the third sensor axis is zero. Each rotation matrix may define the rotation of the respective sensor about the three sensor axes. Calculating the sensor frame estimated gravity vector for each pose associated with each sensor may comprise applying the rotation matrix for each pose associated with each sensor to the gravity vector to transform the direction in which the gravity vector acts to that of the respective pitch and roll angle of the sensor.

The sensor frame estimated gravity vector may be vectors defining the direction along which the gravity vector acts for the respective pitch and roll angle of the sensor.

The method may comprise: receiving a register pose signal which indicates the joint is in one of the poses of the at least two different poses; and in response to the register pose signal, storing the orientation data for each of the two sensors from when the register pose signal is received as the orientation data for that one of the poses of the at least two different poses. The method may comprise repeating the steps of claim 9 for each pose of the at least two different poses.

The orientation data may be associated with four different poses of the joint for each of the two sensors. The poses may be selected from, or are all of, a sitting extension pose, a sitting pose, a standing pose and a standing flexion pose. The estimated joint axis directions may be each three-dimensional vectors in a coordinate system of the respective sensor, the coordinate system may be defined by the three sensor axes of the respective sensor.

The coordinate system may be the sensor frame.

The projections of each sensor frame estimated gravity vector for each pose associated with each sensor on to the estimated joint axis direction for the respective may be calculated by taking the scalar product of the estimated joint axis direction for a particular sensor with the respective sensor frame estimated gravity vector. The loss function may combine each of the projections on to the estimated joint axis direction with the sensor frame estimated gravity vector for one sensor with the projection on to the estimated joint axis direction with the sensor frame estimated gravity vector for the other sensor. The combination of the projections of the two sensors may be taking the difference between the two projections. The loss function may aggregate the combined projections for each pose. The loss function may aggregate the combined projections for each pose by summing together the combined projections. The loss function may aggregate the square of the combined projections for each pose.

The method may comprise calculating an angle of the joint about the joint axis using the estimated joint axis directions for the joint axis for each sensor and orientation data for each of the two sensors.

According to a second aspect of the present invention there is provided a sensor comprising a processor configured to: calibrate respective estimated joint axis directions for each of a pair of body mounted sensors, one of the pair of sensors being located to each side of the joint comprising a joint axis, the sensors each calculating a pitch angle about respective first sensor axes and a roll angle about respective second sensor axes, the first and second sensor axes together with a third sensor axis orthogonal to the first and second sensor axes forming a sensor frame, the device being configured to calibrate the respective estimated joint axis directions by: receiving orientation data for each of the two sensors, the orientation data being associated with at least two different poses of the joint for each of the two sensors and the orientation data comprising the pitch angle and the roll angle of the sensor for each pose; calculating a sensor frame estimated gravity vector for each pose associated with each sensor based on the pitch and roll angles for each pose associated with each sensor and a gravity vector running along a vertical direction; and determining the estimated joint axis directions for the joint axis, relative to the first and second sensor axes, for each sensor that minimise a loss function concerning projections of each sensor frame estimated gravity vector for each pose associated with each sensor on to the estimated joint axis direction for the respective sensors

The present invention will now be described by way of example with reference to the accompanying drawings. In the drawings:

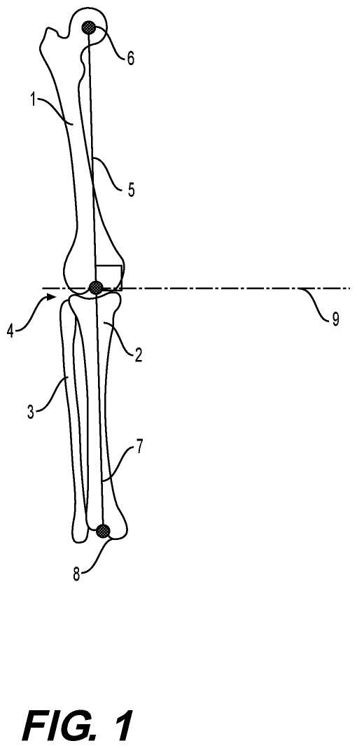

FIG. 1 shows a diagram illustrating a standard leg of a person.

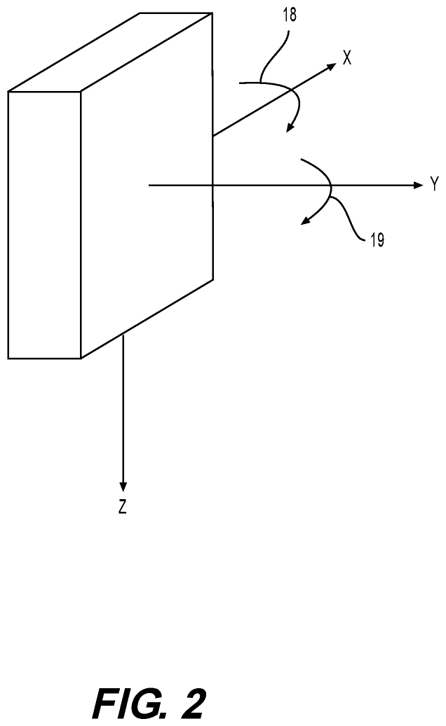

FIG. 2 shows a schematic diagram of a coordinate system associated with the knee joint.

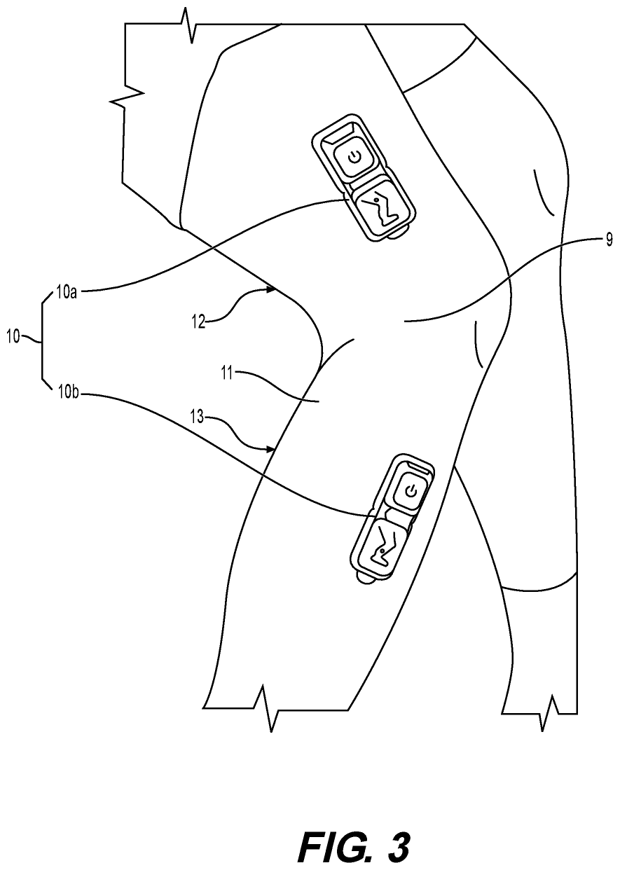

FIG. 3 shows a diagram of a pair of sensors attached to a leg.

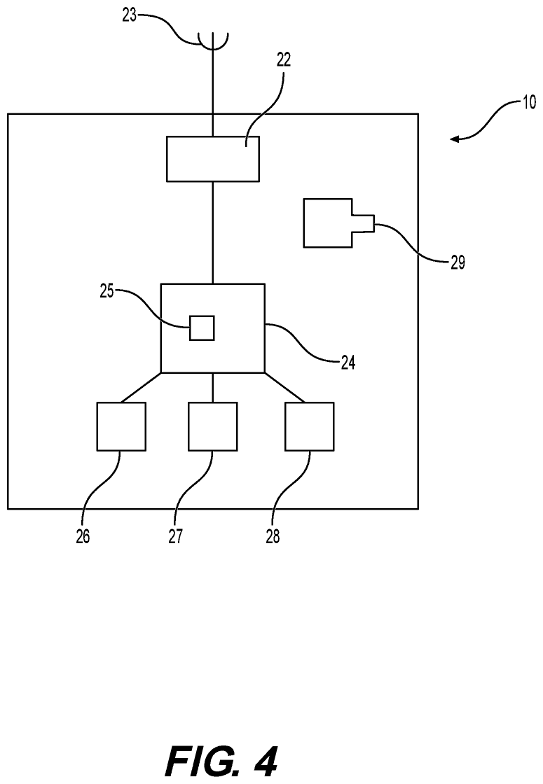

FIG. 4 shows a schematic diagram of a sensor device.

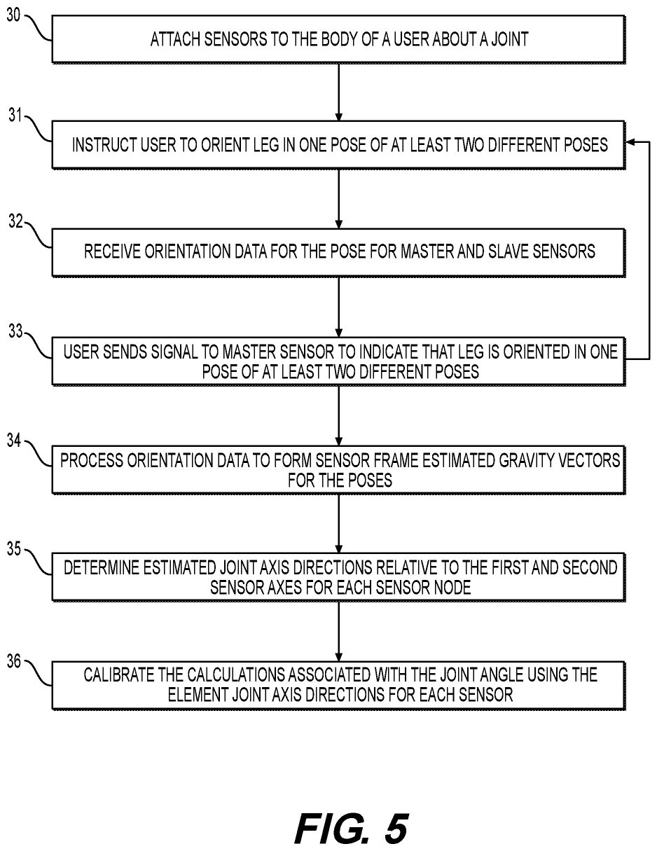

FIG. 5 shows a flow diagram of a method for calculating an estimated direction of a joint axis in the sensor measurement frames.

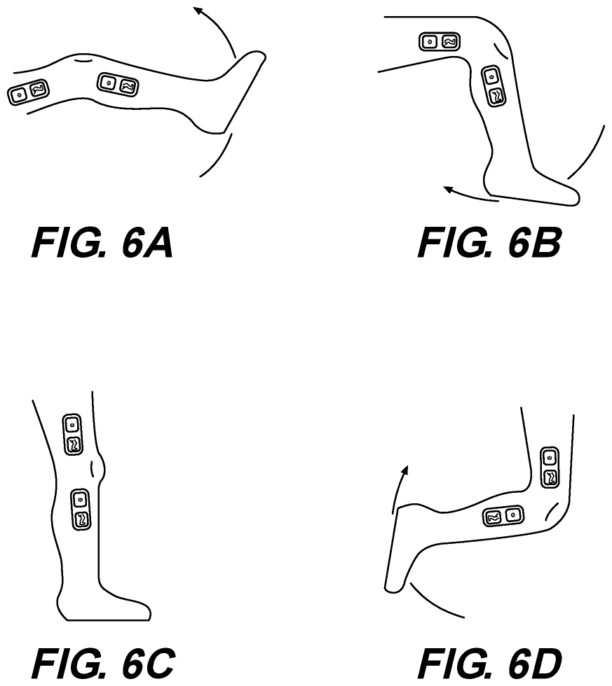

FIG. 6 shows diagrams of poses to be taken by a user of the sensor devices.

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

The present invention relates to a method for calibrating respective estimated joint axis directions for each of a pair of body mounted sensors, one of the pair of sensors being located to each side of the joint comprising a joint axis, the sensors each calculating a pitch angle about respective first sensor axes and a roll angle about respective second sensor axes, the first and second sensor axes together with a third sensor axis orthogonal to the first and second sensor axes forming a sensor frame. The method comprises receiving orientation data for each of the two sensors, the orientation data being associated with at least two different poses of the joint for each of the two sensors and the orientation data comprising the pitch angle and the roll angle of the sensor for each pose. The method further comprises calculating a sensor frame estimated gravity vector for each pose associated with each sensor based on the pitch and roll angles for each pose associated with each sensor and a gravity vector running along a vertical direction. The method further comprises determining the estimated joint axis directions for the joint axis, relative to the first and second sensor axes, for each sensor that minimise a loss function concerning projections of each sensor frame estimated gravity vector for each pose associated with each sensor on to the estimated joint axis direction for the respective sensor. The sensor frame may be a coordinate system of the respective sensor. The coordinate system being defined by the three sensor axes of the respective sensor.

FIGS. 1 and 2 are provided to allow a simple explanation of certain terms that are used within this specification. FIG. 1 illustrates a standard leg of a person, the standard leg having a femur 1, tibia 2 and a fibula 3. These are joined at a knee joint 4. The femur defines a femoral mechanical axis 5 extending from the knee to a ball joint 6 which forms part of the person's hip. A tibial mechanical axis 7 extends from the knee 4 to the lower end 8 of the tibia itself. The femur and the lower leg (made up of tibia 2 and fibula 3) can pivot relative to each other about a knee joint axis 9. The femur and the lower leg thus define a plane in which the respective mechanical axes pivot relative to each other. Thus, each mechanical axis 5, 7 will substantially align with the respective part of the leg, such that the knee joint axis 9 is perpendicular to the plane in which the axes pivot. Thus, the knee joint angle is thus typically the angle between the two mechanical axes. This is an idealised situation, which forms the basic geometry considered by the present invention.

FIG. 2 helps to define the coordinate system associated with the knee joint, as well as how the terms pitch and roll apply to the knee. The convention when discussing the knee joint is that, when a person is standing upright, the x-axis points forward i.e. away from the knee parallel to the ground, the y-axis points to the right of the person, and the z-axis points downwards towards the ground. This convention applies to both left and right legs, i.e. the positive y-axis is always to the right hand side of the knee irrespective of the leg. In a normal knee alignment, the y-axis is therefore analogous to the knee joint axis 9. This can therefore define a global coordinate frame for the knee joint where a first global coordinate frame axis, the y-axis, points to the right of the person parallel to a horizontal floor, a second global coordinate frame axis, the x-axis, points forwards away from the knee and perpendicular to the first global coordinate frame axis, and a third global coordinate frame axis, the z-axis, points downwards towards the ground in a vertical direction. The third global coordinate frame axis being perpendicular to both the first and second global coordinate frame axes.

The orientation of any sensors associated with the knee typically have two components. A rotation of the sensor about the x-axis is a roll motion, identified by arrow 18, and defines a roll angle. A rotation of the sensor about the y-axis is a pitch motion, identified by arrow 19, and defines a pitch angle. Each sensor has its own sensor frame of reference which defines the rotational direction of the sensor about each of the axes.

FIG. 3 illustrates a pair of sensors 10 attached to a leg 11. An upper sensor 10a is placed on the thigh 12 and a lower sensor 10b is placed on the calf 13. The purpose of the sensors is to monitor the flex of the knee at the knee joint, i.e. a pitch angle about the y-axis/knee joint axis 9. If the two sensors 10a, 10b could be aligned such that the z-axis of the sensor was parallel to the respective mechanical axis of the leg, and the sensor y-axis was parallel with the knee joint axis 9, the calculation of the knee angle would be a simple subtraction of the calf pitch angle form the thigh pitch angle. In other variations, more than two sensors could be used. For example, it may be desirable to use three sensors if the joint under surveillance is a ball and socket joint which has three degrees of freedom of movement. In some cases, it may be desirable to use an additional sensor on a joint such as a knee joint to assist in determining orientations of the thigh and calf. For example, two sensors could be placed on one of the user's limbs. Measurements from such a third sensor could be processed in a similar manner to the processing described above for two sensors. One possibility is to process the data from the two sensors placed on one limb to obtain a set of data for that limb, and then process that in conjunction with the data from the other limb.

However, as will be appreciated, the shape and form of a human leg does not generally permit such alignment to be possible, so when the sensors 10a, 10b are in place as shown in FIG. 3, there is a misalignment with the femoral and tibial mechanical axis which needs to be corrected for in order to obtain an accurate knee angle measurement. In particular, as the axes of the sensors 10a, 10b are likely to not be aligned with the axes of the leg and knee, there can be an inaccuracy in the calculated direction of the knee joint axis 9. This can then affect the estimated knee angle depending on the leg shape and positioning of the sensors on the person's thigh and calf. It is therefore important for the sensed angle of the thigh 12 and calf 13 about the knee joint axis 9 and by extension the femoral mechanical axis 5 and the tibial mechanical axis 7 about the knee joint axis 9 to be corrected as much as possible. A method to improve the knee angle estimate for varying leg shapes and sensor positioning by estimating the direction of the knee joint axis is described herein.

FIG. 4 illustrates a schematic diagram of the sensor device 10. The sensor device comprises at least one wireless communication unit 22. The wireless communication unit 22 is connected to an antenna 23.

The sensor device 10 comprises a processor 24 and a non-volatile memory 25. The sensor device 10 may comprise more than one processor 24 and more than one memory 25. The memory 25 stores a set of program instructions that are executable by the processor, and reference data such as look-up tables that can be referenced by the processor in response to those instructions. The processor 24 may be configured to operate in accordance with a computer program stored in non-transitory form on a machine-readable storage medium. The memory 25 may be the machine-readable storage medium. The computer program may store instructions for causing the processor to perform the method described herein.

The processor 24 may be connected to the wireless communication unit(s) 22 to permit communication between them. The processor 24 may use at least one wireless communication unit to send and/or receive data over a wireless communication network. For instance, the wireless communication unit(s) 22 may be:
A cellular communication unit configured to send and receive data over a cellular communication network. The cellular communication unit may be configured to communicate with cellular base stations to send and receive data.
A Wi-Fi communication unit configured to send and receive data over a wireless communication network such as a Wi-Fi network. The Wi-Fi communication unit may be configured to communicate with wireless base stations to send and receive data.
A Bluetooth communication unit configured to send and receive data over a Bluetooth communication network. The Bluetooth communication network may be configured to communicate with other Bluetooth devices to send and receive data.

It will be appreciated that the wireless communication unit(s) may be configured to communicate using other wireless protocols.

One or more of the wireless communication units 22 may be part of processor 24. Part or all of a wireless communication unit's function may be implemented by processor 24 by processor running software to process signals received by an antenna 23.

The sensor device 10 may use the wireless communication unit(s) 22 to communicate between the sensor device 10 and another sensor device 10 to share information concerning the sensed rotation of the sensor device 10 with the other sensor device 10. In this case, the sensor devices 10 may make use of a short range communication protocol such as Bluetooth or Zigbee. The sensor device 10 may also communicate with another form of device such as a smartphone. This communication may be used to share data concerning the knee angle estimates over time either in the form of aggregated data collected over time or by streaming live knee angle estimates to the smartphone as they are calculated. In this case, the sensor device 10 may use a short-range communication protocol such as Bluetooth or Zigbee if the other device is located nearby, or a longer-range communication protocol such as Wi-Fi or even cellular-based communications.

The sensor device 10 may comprise a power source 29 such as a battery. The sensor device 10 may accept an external power supply to enable the power source 29 to be charged. The sensor device 10 may be wirelessly chargeable. The sensor device 10 may also comprise a display. The sensor device 10 may be configured to display information on the display. The sensor device 10 may also comprise a user interface. The user interface may be configured to permit a user of the device to interact with the sensor device 10. The user interface may at least in part be formed as part of the display. For instance, the display may be a touch screen and display buttons and other interactive features of the display that the user can interact with by touching the touch screen.

The sensor device 10 comprises at least one movement sensor. The processor 24 is connected to the movement sensors to permit communication between them. The processor 24 can receive movement data from the movement sensors. The movement sensors may comprise at least one accelerometer, a magnetometer, and/or a gyroscope. The processor 24 can use the movement data from the movement sensors to derive information about the current movement and, in particular, the current orientation of the sensor device 10. Advantageously, the sensor device 10 comprises a triaxial accelerometer and a gyroscope. Some or all of the movement sensors may be packaged together in an inertial measurement unit (IMU).

The mobile device comprises at least one accelerometer 26. The accelerometer may calculate the acceleration rate that the device 10 is being moved in a direction. The accelerometer may output time series data of acceleration readings in the direction that the accelerometer 26 gathers data. The device 10 may comprise more than one accelerometer 26. The accelerometers 26 may be orientated so as to gather acceleration readings in different directions. The accelerometers 26 may gather acceleration readings in orthogonal directions. The device may comprise three accelerometers 26 each gathering acceleration readings in different, orthogonal directions, thus the device may comprise a triaxial accelerometer. The processor 4 can receive the time series data of acceleration readings from the at least one accelerometer.

The mobile device may comprise a magnetometer 27. The magnetometer may calculate the orientation of the device 10 relative to the local magnetic field of the Earth. This can be used to derive data concerning the movement of the device 10 relative to the surface of the Earth. The magnetometer may be a hall effect sensor that detects the magnetic field of the Earth. The magnetometer 27 may output time series data of rotation movement readings relative to the magnetic field of the Earth. The processor 24 can receive the time series data of rotation movement readings.

The mobile device 10 comprises a gyroscope 28. The gyroscope 28 may be a MEMS gyroscope. The gyroscope 28 may calculate the rate of rotation about a rotation axis that the device 10 is being moved about. The gyroscope 28 may output time series data of rotation rate readings about the rotation axis that the gyroscope 28 gathers data. The time series data of rotation rate readings may be rotation rate data. The gyroscope 28 may gather data about the rate of rotation about more than one rotation axis that the device 10 is being moved about. The gyroscope 28 may calculate the rate of rotation about two rotation axes that the device 10 is being moved about. The gyroscope 28 may calculate the rate of rotation about three rotation axes that the device 10 is being moved about. Thus, the gyroscope 28 may be a triaxial gyroscope. The rotation axes may be orthogonal to each other. The gyroscope 28 may output time series data of rotation rate readings about each rotation axis that the gyroscope 28 gathers data. The time series comprise rotation reading(s) at each time step in the time series. The processor 24 can receive the time series data of rotation rate readings about one or more axes.

As discussed herein, two sensor devices 10a, 10b are used to calculate an estimate of knee angle at each time step. One sensor device 10a acts as a master sensor device and one sensor device 10b acts as a slave sensor device. The slave sensor device 10b sends orientation data for each time step to the master sensor device 10a. The master sensor device 10a then processes the slave's orientation data together with its own orientation data to estimate the knee angle at each time step. The orientation data is sent from the slave to the master using the wireless communication units 22 present in each sensor device 10a, 10b.

The orientation data comprises a pitch angle and a roll angle that has been sensed by the sensor device 10 at a particular time step. The pitch angle sensed by the sensor device 10 is about a first sensor axis. This first sensor axis may be known as a pitch axis. The roll angle sensed by the sensor device 10 is about a second sensor axis. This second sensor axis may be known as a roll axis. Each sensor device 10 senses its orientation (and thus rotation) about their own respective first and second sensor axes. The first and second sensor axes are orthogonal to each other. There is a third sensor axis about which the sensor 10 can move. This sensor axis is orthogonal to the first and second sensor axes. In the case of a knee joint, the sensors may be attached to the body about the joint so that the third sensor axis runs in a generally vertical direction when the user is standing up with the leg fully extended. Thus, in this position the third sensor axis may run generally along the third global coordinate frame axis. The sensors may be attached to the body so that the first sensor axis runs generally parallel to the joint axis, however as described herein there may be some difference between the joint axis and the sensor axis which needs to be corrected for. The sensors may be attached to the body so that the second sensor axis points in a forward direction and runs perpendicular to the first sensor axis. The three sensor axes define the sensor frame of reference.

The pitch and roll angles are derived from the data calculated by the movement sensors. For instance, data from the accelerometers and the gyroscope may be combined to give the current pitch and roll angles of the sensor device 10. The sensor device 10 may use current and historic data from the movement sensors to derive the current pitch and roll angles for the sensor device 10. The method by which the pitch and roll angles are calculated may use any conventional method. By way of example, one such method is described in “Estimation of IMU and MARG orientation using a gradient descent algorithm” S. Madgwick et al, 2011 IEE International Conference on Rehabilitation Robotics, Rehab Week Zurich Science City, Switzerland, Jun. 29-Jul. 1, 2011.

A method for calculating an estimated direction of the joint axis in the sensor measurement frames will now be described with reference to FIG. 5. This method may be undertaken by processor 4 in one of the pair of sensor devices 10. Whilst the example description refers to a knee and a leg in places, it will be appreciated that other joints could also be monitored in this way.

As shown at step 30, the sensors are attached to the body of a user about a joint. The joint has a joint axis and one of the pair of sensors is located to each side of the joint. The sensors are switched on and paired together so that one of the sensors 10b sends its orientation data to the other sensor 10a. As discussed herein, the sensor that sends data to the other sensor is a slave sensor 10b and the sensor that receives data from the other sensor is a master sensor 10a.

As shown at step 31, the user is instructed to orient the leg that has the sensors attached to it into one of the poses of at least two different poses. As shown at step 32, the master sensor receives orientation data for the pose from both itself and the slave sensor. In the case of the master sensor, it may receive the orientation data from a separate process running on the processor 4 which takes the movement sensor raw data and processes it to get the orientation data for that pose. As shown at step 33, the user sends a signal to the master sensor to indicate that the leg has been oriented in one of the poses of at least two different poses and in response to this signal the master sensor stores the orientation data as being associated with that particular pose. This signal may be sent to the master sensor by pressing a button on the master sensors. Alternatively, the master sensor may be in communication with another device, such as a smartphone, and the signal is sent from the other device to the master sensor. The user may have pressed a button on the other device to cause the signal to be sent to the master sensor. The process of steps 31 to 33 are repeated until the orientation data from both sensors for each of the poses has been received by the master sensor.

The poses that the user is instructed to put the leg in are used to orient the sensors in different directions to enable an estimate of the joint axis, as seen by each sensor, to be determined. The poses are chosen so that each pose gives some different information about the rotation of the sensors relative to the joint axis. For instance, the sensors are placed in the same or different rotational positions relative to each other so that the rotation axis of the joint runs in particular directions relative to the sensor position at in a given pose.

Advantageously, the user is instructed to orient the leg in four poses. These poses are shown in FIG. 6. A first pose is shown in FIG. 6A. In the first pose, the user is instructed to extend their leg as much as possible in a horizontal direction so that the thigh and calf are oriented as generally parallel as possible in a horizontal direction. The user is likely to be sitting down to achieve this pose. This first pose may be known as a sitting extension pose. A second pose is shown in FIG. 6B. In the second pose, the user is instructed to bend the leg so that the thigh runs generally horizontal and the calf runs generally vertical. The user is likely to be sitting down to achieve this pose. This second pose may be known as a sitting pose. A third pose is shown in FIG. 6C. In the third pose, the user is instructed to extend their leg as much as possible in a vertical direction so that the thigh and calf are oriented as generally parallel as possible in a vertical direction. The user is likely to be standing up to achieve this pose. This third pose may be known as a standing pose. A fourth pose is shown in FIG. 6D. In the fourth pose, the user is instructed to bend the leg so that the thigh runs generally vertical and the calf runs generally horizontal. This fourth pose may be known as a standing flexion pose. In the case of other limbs and joints, the lower and upper parts of the limb to either side of the joint may be orientated as for the thigh and calf described herein for each pose. As the user's knee, or other joint, may have limited movement after surgery or injury, the user is instructed to move the leg, or other limb, into as close to these poses as the user is able to.

Once the orientation data for each pose has been received from each of the two sensors, the orientation data can be processed to form estimated gravity vectors in the sensor frames for the poses. This is as shown in step 34. A sensor frame estimated gravity vector gives the orientation of the sensor relative to the gravity vector that would be recorded by the sensor based on the current rotation of the sensor about the roll and pitch axes assuming that gravity acts along a vertical direction (i.e. along the third global coordinate frame axis). Thus, the sensor frame estimated gravity vector may be based on the pitch and roll angles and a gravity vector running along a vertical direction. The sensor frame estimated gravity vectors are three dimensional vectors.

As described here, the orientation data for each pose from each sensor comprises a pitch angle and a roll angle. These describe the orientation of each sensor whilst the leg is in a particular pose. A rotation matrix is formed for each associated pitch angle and roll angle. I.e. there is one rotation matrix formed for the pitch angle and roll angle recorded for a particular pose by one of the sensors. Therefore, a rotation matrix is formed for each pair of pitch and roll angles associated with a respective pose for a respective sensor. The rotation matrix defines the rotation of the sensor about the three sensor axes. As only the roll and pitch measurements are important for calculating the knee joint angle, it is assumed that there is no rotation about the third sensor axis. An example rotation matrix for a pitch angle of θ and a roll angle of α about the first and second sensor axes respectively is:

$${R}_{i} = [ \begin{matrix}cos θ & 0 & sin θ \\ sin θ sin α & cos α & - sin α cos θ \\ - sin θcos α & sin α & cos θcos α\end{matrix} ]$$

where Rᵢ is the sensor i rotation matrix formed from the pitch and roll angles for a particular pose, i is either the first or second sensor devices, θ is the pitch angle, and α is the roll angle.

The rotation matrices are used to form the sensor frame estimated gravity vectors. This uses the assumption that gravity acts in a vertical direction and thus along the third global coordinate frame axis. In the example given herein, the third global coordinate frame axis is assumed to point towards the ground meaning that the acceleration due to gravity acts in an upward (negative) direction. The rotation matrices act upon the gravity vector to produce the sensor frame estimated gravity vectors. The rotation matrices rotate the gravity vector using the roll and pitch angles to calculate the direction in which gravity acts on the sensor whilst in that orientation defined by the roll and pitch angles. The sensor frame estimated gravity vectors may be calculated by:

aᵢ=Rᵢ^{T}g

where aᵢ is the sensor frame estimated gravity vector for sensor i based on particular roll and pitch angles, Rᵢ^{T} is the transpose of the sensor i rotation matrix and g is the gravity vector g=[0, 0, −9.81]^{T}. i is either the master (M) or slave (S) sensor device to which the roll and pitch angles relate.

The use of the rotation matrices to transform the gravity vector to produce the sensor frame estimated gravity vector are advantageous because they only depend on the pitch and roll angles. These have been derived from the motion sensors inside of the sensor devices and so are based on more data than an accelerometer on its own can provide and so should provide a more accurate value for the pitch and roll angles. This then follows that the sensor frame estimated gravity vectors should also be more accurate than using accelerometer readings directly. This compound calculation to produce the pitch and roll angles also means that there is less dependency on the user being static at each pose than if the accelerometer outputs were used directly. This compound calculation to produce the pitch and roll angles also means that there is less dependence on the user being able to move quickly than if a gyroscope output was used directly. In addition, by converting the pitch and roll angles to rotations and then to sensor frame estimated gravity vectors less data is required to input into the loss function making it more efficient to calculate. This thus provides advantages over the method described in “On motions that allow for identification of hinge joint axes from kinematic constraints and 6D IMU data”, Danny Nowka et al, available at https://www.control.tu-berlin.de/wiki/images/b/b3/Nowka2019_ECC.pdf.

As shown in step 35, estimated joint axis directions relative to the first and second sensor axes for each sensor device 10 are determined. The estimated joint axis directions are each three-dimensional vectors in the coordinate system of the respective sensor device. The coordinate system of the sensor device being defined by the three sensor axes. The estimated joint axis directions are determined by finding the joint axis directions that minimise a loss function concerning the projections of each sensor frame estimated gravity vector for each pose associated with each sensor on to the estimated joint axis direction for the respective sensor. The sensor frame estimated gravity vectors are protected on to the estimated joint axis direction. This projection may involve taking the scalar product of the estimated joint axis direction for a particular sensor with the sensor frame estimated gravity vector for a particular sensor associated with a particular pose. The loss function may combine the projection on to the estimated joint axis direction with the sensor frame estimated gravity vector for one sensor with the projection on to the estimated joint axis direction with the sensor frame estimated gravity vector for the other sensor. The combination of the projections of the two sensors may be the difference between the two projections.

The loss function may combine together the combined projections for each pose. In other words the loss function aggregates the combined projections for each pose. This combination may be the sum of the combined projections. The combination of the combined projections for each pose may involve combining the square of the combined projections for each pose together. The square of each of the combined projections may be summed together. Instead of the square of the combined projections, the loss function may take the magnitude of the combined projections.

The loss function may be calculated by the equation:

$$L = {\sum }_{k = 1}^{N} {( {j}_{M}^{T} {a}_{M}^{k} - {j}_{S}^{T} {a}_{S}^{k} )}^{2}$$

where L is the loss function, jᵢ is the estimated joint axis direction for sensor i, a_{M}^{k} is the sensor frame estimated gravity vector for sensor i in pose k, k are the poses for which orientation data has been recorded, and N is the number of poses. In the advantageous example described herein, the number of poses may be four.

The estimated joint axis directions for each sensor that provide the minimum of the loss function may be determined by any relevant method. For instance, an iterative approach may be used to approach the minimum value for the loss function whilst varying the direction of the two estimated joint axes.

As shown at step 36, once the estimated joint axis directions for each sensor have been determined, the master sensor device 10a can use these estimated joint axis directions to calibrate the calculations associated with the joint angle. The master sensor device receives orientation data from the slave device and also its own orientation processing section. The roll and pitch angles comprised in the orientation data for each time step can be transformed based on the estimated joint axis directions to determine the rotation of each of the two sensors about the estimated joint axis direction. The master device can then take the difference between the angle of one of the sensor device relative to the other to determine the current knee joint angle about the joint axis. Corrections to the calculated knee joint angle may be made to account for misplacement of the sensors.

The above method therefore provides the advantage of providing a correction method to make the calculation of the knee joint angle, or other joint angle, more accurate. This can improve the accuracy of the data gathered by these devices and thus permit better analysis of the movement of the joint.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A method for calibrating estimated joint axis directions for each of a pair of sensors, one of the pair of sensors being mounted to each side of a joint comprising a joint axis, each sensor of the pair of sensors calculating a pitch angle about a first sensor axis and a roll angle about a second sensor axis, the first sensor axis and second sensor axis together with a third sensor axis orthogonal to the first sensor axis and the second sensor axis forming a sensor frame, the method comprising:
receiving orientation data for each of the pair of sensors, the orientation data being associated with at least two different poses of the joint for each of the pair of sensors and the orientation data comprising the pitch angle and the roll angle of the sensor for each pose;
calculating a sensor frame estimated gravity vector for each pose associated with each sensor based on the pitch and roll angles for each pose associated with each sensor and a gravity vector running along a vertical direction; and
determining the estimated joint axis directions for the joint axis, relative to the first sensor axis and the second sensor axis, for each sensor that minimise a loss function concerning projections of each sensor frame estimated gravity vector for each pose associated with each sensor on to the estimated joint axis direction for the respective sensor.

2. The method according to claim 1, wherein the gravity vector runs along the vertical direction in a negative direction.

3. The method according to claim 1, wherein the gravity vector runs along the vertical direction in an upward direction.

4. The method according to claim 1, wherein calculating the sensor frame estimated gravity vector for each pose associated with each sensor comprises forming a rotation matrix for each pose associated with each sensor using the pitch angle and the roll angle for each pose associated with each sensor.

5. The method according to claim 4, wherein forming the rotation matrix for each pose associated with each sensor using the pitch and roll angles for each pose associated with each sensor comprises assuming the rotation about the third sensor axis is zero.

6. The method according to claim 4, wherein each rotation matrix defines the rotation of the respective sensor about the first sensor axis, the second sensor axis, and the third sensor axis.

7. The method according to claim 4, wherein calculating the sensor frame estimated gravity vector for each pose associated with each sensor comprises applying the rotation matrix for each pose associated with each sensor to the gravity vector to transform the direction in which the gravity vector acts to that of the respective pitch and roll angle of the sensor.

8. The method according to claim 1, wherein the sensor frame estimated gravity vector are vectors defining the direction along which the gravity vector acts for the respective pitch and roll angle of the sensor.

9. The method according to claim 1, the method comprising:
receiving a register pose signal which indicates the joint is in one of the poses of the at least two different poses; and
in response to the register pose signal, storing the orientation data for each of the pair of sensors from when the register pose signal is received as the orientation data for that one of the poses of the at least two different poses.

10. The method according to claim 9, the method comprising repeating the steps of claim 9 for each pose of the at least two different poses.

11. The method according to claim 1, wherein the orientation data is associated with four different poses of the joint for each of the pair of sensors.

12. The method according to claim 1, wherein the poses are selected from, or are all of, a sitting extension pose, a sitting pose, a standing pose and a standing flexion pose.

13. The method according to claim 1, wherein the estimated joint axis directions are each three-dimensional vectors in a coordinate system for each sensor of the pair of sensors, the coordinate system being defined by the first sensor axis, the second sensor axis, and the third sensor axis for each sensor of the pair of sensors.

14. The method according to claim 1, wherein the projections of each sensor frame estimated gravity vector for each pose associated with each sensor on to the estimated joint axis direction for the respective are calculated by taking a scalar product of the estimated joint axis direction for a particular sensor with the respective sensor frame estimated gravity vector.

15. The method according to claim 1, wherein the loss function combines each of the projections on to the estimated joint axis direction with the sensor frame estimated gravity vector for one sensor of the pair of sensors with the projection on to the estimated joint axis direction with the sensor frame estimated gravity vector for a remaining sensor of the pair of sensors.

16. The method according to claim 15, wherein the combination of the projections of the pair of sensors is taking a difference between the projections.

17. The method according to claim 15, wherein the loss function aggregates the combination of the projections for each pose.

18. The method according to claim 17, wherein the loss function aggregates the combination of the projections for each pose by summing together the combined projections.

19. The method according to claim 17, wherein the loss function aggregates a square of the combination of the projections for each pose.

20. The method according to claim 1, the method comprising calculating an angle of the joint about the joint axis using the estimated joint axis directions for the joint axis for each sensor and orientation data for each of the pair of sensors.

21. (canceled)
